(19) United States
(12) Patent Application Publication
(10) Pub. No.: US 2014/0065078 A1
(43) Pub. Date: Mar. 6, 2014

(54) ORAL CARE COMPOSITION FOR PROMOTING AND MAINTAINING ORAL HEALTH AND METHOD OF FORMING AND USING SAME

(71) Applicant: Dr. Fresh, LLC, Buena Park, CA (US)

(72) Inventor: Puneet Nanda, Cerritos, CA (US)

(73) Assignee: Dr. Fresh, LLC, Buena Park, CA (US)

(21) Appl. No.: 14/017,149

(22) Filed: Sep. 3, 2013

Related U.S. Application Data
(60) Provisional application No. 61/696,100, filed on Aug. 31, 2012.

(51) Int. Cl. A61K 8/66; A61Q 11/00

(52) U.S. Cl. CPC A61K 8/66; A61Q 11/00
USPC 424/50

(57) ABSTRACT

An oral care composition, a method of forming the oral care composition, and a method of using the oral care composition are disclosed. The oral care composition includes a plurality of enzymes to prevent formation of and/or facilitate the breakup of biofilm in an oral cavity and a metal ion management system to inhibit growth of gram negative bacteria.

### CROSS REFERENCE TO RELATED APPLICATIONS

This application claims the benefit of Provisional Application Ser. No. 61/696,100, filed Aug. 31, 2012, entitled ORAL CARE COMPOSITION FOR PROMOTING AND MAINTAINING ORAL HEALTH AND METHOD OF FORMING AND USING SAME.

### FIELD OF THE INVENTION

The present invention generally relates to compositions and methods for promoting and maintaining oral health.

### BACKGROUND OF THE INVENTION

Maintaining oral health is generally important for a variety of reasons. If oral health is not maintained, a variety of conditions, ranging from bad breath to general health problems, may occur. Such conditions include, for example, tooth decay, tooth coloration, gum disease, tooth loss, and even general health problems, such as heart disease, stroke, poorly controlled diabetes and preterm labor.

Typical compositions for maintaining or promoting oral health often include one or more relatively strong antibacterial and/or antimicrobial agents. Although such agents may work relatively well to reduce an amount of bacteria or microbes in an oral cavity, use of strong antimicrobial agents may be undesirable or even detrimental. For example, application of relatively large doses of such agents may be disadvantageous, because, in addition to destroying “bad” or undesirable bacteria and/or microbes, the agents may also kill good (e.g., “healthy” or “normal”) bacteria and/or microbial agents within the oral cavity.

In general, it may be desirable to maintain a healthy flora (i.e., normal levels of certain bacteria and other microbials), rather than a reduced flora. In addition, other formulations for oral care treatment are said to have a bad taste or stain tooth enamel. Accordingly, improved oral compositions and methods of making and using the compositions are desired.

### SUMMARY OF THE INVENTION

The present invention provides an improved oral care composition, a method of using the composition, and a method of forming the composition. As set forth in more detail below, the composition can be used to treat a variety of conditions, including providing soothing relief to at least a portion of an oral cavity, treating or preventing dry mouth, developing and/or maintaining a healthy or normal flora in an oral cavity, reducing an amount of biofilm in the oral cavity, treating or preventing bad breath, treating common colds, reducing tartar formation, reducing plaque formation, reducing gum and/or periodontal disease, and the like. The ways in which the present invention addresses various drawbacks of now-known oral compositions and methods are discussed in greater detail below. However, in general, the present invention provides a composition for selectively attacking and/or mitigating growth of certain bacterial and microbial compounds, while allowing other bacterial and microbial compounds to survive, which, in turn, promotes oral health.

In accordance with various exemplary embodiments of the present invention, a composition includes a plurality of enzymes to promote healthy flora in an oral cavity and a metal ion management formulation. The plurality of enzymes may, for example, attack undesired bacteria and/or reduce inflammation within an oral cavity. The plurality of enzymes may include two or more enzymes selected from the group consisting of: lysozyme, serrapeptase (e.g., Peptizyme®-SP) or serratiopeptidase, papain, and optionally amylases, glycosidase, dextrinase, and/or amylogucosidase. In accordance with particular aspects, the composition includes lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase.

In accordance with further exemplary aspects of these embodiments, the metal ion management formulation includes one or more compounds that bind with iron and other metal ions to inhibit growth of gram negative bacteria. Exemplary compounds suitable for inhibiting growth of gram negative bacteria include sodium EDTA, phytic acid, lactoferrin, and various combinations thereof. In accordance with further aspects of these embodiments, a composition includes additional actives, such as a zinc salt, e.g., zinc gluconate or zinc chloride (plaque/tartar reduction agent), a demulcent, xylitol (caries inhibition agent), other anticaries actives, such as sodium fluoride, desensitizing agents, such as potassium nitrate, and/or aloe vera (soothing agent). In accordance with yet further aspects, the composition includes a carrier, which may include solvents, sweeteners, preservatives, thickeners and/or emulsifiers, surfactants, flavorings, pH stabilizers, and the like. The composition may be in a variety of configurations, such as an oral rinse, an oral spray, a foaming liquid, liquid drops, strips, gums, lozenges or the like. Exemplary compositions may comprise, consist of, or consist essentially of an actives formulation that comprises, consists of, or consists essentially of the active ingredients noted herein and a carrier composition that comprises, consists of, or consists essentially of the other or inactive ingredients noted herein.

In accordance with further embodiments, a composition is configured for use by people with diabetes or prediabetes, such that none of the ingredients are contraindicated for diabetics or prediabetics. For example, the active ingredients, such as enzymes, may be selected such that the enzymes do not break down carbohydrates into sugars. One of the most common oral health problems for diabetics is xerostomia and the lack of saliva flow is believed to be the primary reason for the increased incidence of gum disease among those that suffer from diabetes. Saliva has several important functions. Saliva prevents or mitigates infections by controlling bacteria in the oral cavity. Saliva also moistens and cleanses the mouth by neutralizing acids produced by dental plaque, and saliva washes away dead skin cells and food debris that can accumulate on gums, the tongue and cheeks within the oral cavity.

In accordance with additional embodiments of the invention, a method of using the composition includes the steps of applying a dose (e.g., about four teaspoons or 20 ml) of the composition including a plurality of enzymes selected from the group consisting of: lysozyme, serrapeptase (e.g., Peptizyme®-SP) or serratiopeptidase, papain, and optionally amylases, glycosidase, dextrinase, and/or amylogucosidase (e.g., the composition may include lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase) and a metal ion management formulation to an oral cavity, maintaining the composition in the oral cavity for an extended period of time (e.g., about 30 seconds to about one minute), spitting out or removing excess composition from the oral cavity, and repeating the process 2-3 times per day or as needed. In accordance with various aspects of these embodiments, the method of using the composition includes general promotion of oral health, providing soothing relief to at least a portion of an oral cavity, prevention and/or reduction of bad breath, prevention and/or reduction of tartar and/or plaque buildup, and/or reduction of gingivitis, gum disease, and/or periodontal disease, prevention and/or reduction of dry mouth, treatment of a common cold, combinations thereof or the like. The composition may be used as desired, used prior to going to bed and/or used after eating.

In accordance with yet additional embodiments of the invention, a method of using the composition includes the steps of spaying a dose of the composition including a plurality of enzymes selected from the group consisting of: lysozyme, serrapeptase (e.g., Peptizyme®-SP) or serratiopeptidase, papain, and optionally amylases, glycosidase, dextrinase, and/or amylogucosidase (e.g., the composition may include lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase) and a metal ion management formulation to an oral cavity, maintaining the composition in the oral cavity for an extended period of time (e.g., about 30 seconds to about one minute), spitting out or removing excess composition from the oral cavity, and repeating the process 2-3 times per day or as needed. In accordance with various aspects of these embodiments, the method of using the composition includes general promotion of oral health, providing soothing relief to at least a portion of an oral cavity, prevention and/or reduction of bad breath, prevention and/or reduction of tartar and/or plaque buildup, prevention and/or reduction of gingivitis, gum disease, and/or periodontal disease, prevention and/or reduction of dry mouth, treatment of a common cold, or the like. The composition may be used as desired, used prior to going to bed and/or used after eating.

In accordance with additional embodiments of the invention, a method of using the composition includes applying a foaming liquid including a plurality of enzymes selected from the group consisting of: lysozyme, serrapeptase (e.g., Peptizyme®-SP) or serratiopeptidase, papain, and optionally amylases, glycosidase, dextrinase, and/or amylogucosidase (e.g., the composition may include lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase) and a metal ion management formulation to an interior portion of an oral cavity, such as on a tongue. In accordance with various aspects of these embodiments, the method of using the composition includes general promotion of oral health, providing soothing relief to at least a portion of an oral cavity, prevention and/or reduction of bad breath, prevention and/or reduction of tartar and/or plaque buildup, and/or reduction of gingivitis, gum disease, and/or periodontal disease, prevention and/or reduction of dry mouth, treatment of a common cold, or the like. The composition may be used as desired, used prior to going to bed and/or used after eating.

In accordance with additional embodiments of the invention, a method of using the composition includes applying a liquid drop formulation (e.g., about 2-3 drops, each drop about 0.025 ml) including a plurality enzymes selected from the group consisting of: lysozyme, serrapeptase (e.g., Peptizyme®-SP) or serratiopeptidase, papain, and optionally amylases, glycosidase, dextrinase, and/or amylogucosidase (e.g., the composition may include lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase) and a metal ion management formulation to an interior portion of an oral cavity, such as on a tongue. In accordance with various aspects of these embodiments, the method of using the composition includes general promotion of oral health, providing soothing relief to at least a portion of an oral cavity, prevention and/or reduction of bad breath, prevention and/or reduction of tartar and/or plaque buildup, and/or reduction of gingivitis, gum disease, and/or periodontal disease, prevention and/or reduction of dry mouth, treatment of a common cold, or the like. The composition may be used as desired, used prior to going to bed and/or used after eating.

In accordance with yet additional embodiments of the invention, a method of forming the composition includes the steps of adding a diluent/solvent (e.g., purified water) to a mixing vessel, mixing in sweetener(s) (e.g., xylitol, stevia), a soothing agent or moisturizer (e.g., aloe vera), an active (e.g., a zinc salt, such as zinc gluconate or zinc chloride), extracting some of the mixture and adding an ion management compound (e.g., lactoferrin) and enzymes to the extracted mixture and transferring the composition back to the mixer. Then, a surfactant/thickening agent (e.g., concentrated cocamidopropyl betaine) is mixed into the composition. Next, in a separate vessel, a premixture of a demulcent and a thickening agent is mixed until the thickener is completely dispersed and there are no visible lumps. The premixture is then added to the mixture. Additional demulcent is then mixed in. Flavoring is then added to the mixture.

### DETAILED DESCRIPTION OF EXEMPLARY EMBODIMENTS

The present invention provides an improved oral care composition and methods of forming and using the composition. As set forth in greater detail below, the oral care composition, in accordance with various embodiments of the invention, includes enzymes to selectively reduce unwanted bacteria, selectively inhibit the growth of unwanted bacteria, reduce an amount of biofilm present in an oral cavity, provide soothing relief, and/or reduce inflammation, plaque, tartar, gingivitis, and/or periodontal disease. The oral care composition also facilitates or increases the moisture retention capabilities of oral mucous and reduces irritation in the oral cavity. The composition can be used for treatment and/or prevention of a variety of conditions, such as tartar reduction, to provide soothing relief, or to treat dry mouth, common colds, bad breath, or the like, and can be used in the treatment of various animals, and is particularly well suited for the treatment of humans.

In accordance with various embodiments of the invention, the oral care composition may be used to treat and/or prevent a variety of conditions of those suffering diabetes or prediabetes. In this case, the composition may desirably not include ingredients contraindicated for use by or treatment of diabetics or prediabetics. In these cases, enzymes that break down carbohydrates into sugars may be avoided in the composition.

By way of particular examples, the oral care composition can be used to promote oral health, provide soothing relief, treat dry mouth, treat bad breath, and/or reduce inflammation, plaque, tartar, and gum and/or periodontal disease in patients with diabetes or prediabetes.

In accordance with various embodiments of the invention, a composition includes a plurality of enzymes that prevent formation of and/or break up of biofilm in an oral cavity and a metal ion management formulation. In accordance with aspects of these embodiments, the enzymes and other ingredients are not contraindicated for use by or treatment of those suffering from diabetes or prediabetes.

The plurality of enzymes may include two or more enzymes selected from the group consisting of: lysozyme, serrapeptase (e.g., Peptizyme®-SP) or serratiopeptidase, papain, and optionally amylases, glycosidase, dextrinase, and/or amylogucosidase. In accordance with particular aspects, the composition includes lysozyme, serrapeptase (e.g., Peptizyme®-SP) or serratiopeptidase, and papain or lysozyme, serrapeptase (e.g., Peptizyme®-SP) or serratiopeptidase, papain, glycosidase, dextrinase, and amylogucosidase.

Lysozyme is a naturally occurring protein that attacks cell walls of certain bacteria (e.g., Clostridia bacteria). Lysozyme gradually weakens the walls of certain cells, causing the cells to eventually burst.

Serrapeptase and serratiopeptidase are anti-inflammatory enzymes that reduce dental infections and can help reduce an amount of plaque on a surface within an oral cavity.

Papain is a protease that helps reduce plaque on a surface of an oral cavity and also helps whiten teeth.

Amylases and amyloglucosidase are dextrinizing enzymes, which can break down complex foods containing sugars. Sugar in the mouth may lead to cavities, so breaking down the foods containing sugars helps reduce cavities and growth of unwanted bacteria.

Glucoxidase is a naturally occurring microbial enzyme that reacts with available oxygen to deprive aerobic bacteria of oxygen and thereby interfere with the aerobic bacteria growth.

Dextrinase is an enzyme that catalyzes the hydrolysis of dextrins.

In accordance with various embodiments, the metal ion management formulation comprises one or more of: phytic acid, lactoferrin, and sodium EDTA. The metal ion management formulation is thought to be a basis for iron and other essential metal ion deprivation, which selectively inhibits growth of gram negative (“bad”) bacteria.

Lactoferrin is an iron binding glycoprotein, which is thought to react with iron required for pathogenic bacteria growth, destroying bacteria through iron deprivation. Phytic acid and sodium EDTA are chelating agents which bind to iron, to thereby inactivate the iron.

The composition of the present invention also includes a suitable carrier and may include other actives. In accordance with exemplary embodiments of the invention, the carrier includes one or more solvents or diluents such as purified water and propanediol, one or more sweeteners and/or sugar alcohols, such as stevia and xylitol (also caries reducing), buffering agents, such as disodium phosphate, a moisturizer or soothing agent, such as aloe vera (which may alternatively be an active), thickeners or emulsifiers, such as carboxymethylcellulose, cellulose gum, polyvinyl pyrolidone, concentrated cocamidopropyl betaine (Tegobetain F 50), or hydroxylethyl cellulose, pH stabilizers, such as disodium phosphate, a preservative such as sodium benzoate and benzoic acid, and natural flavorants, such as natural, mild cinnamon, mint and/or menthol.

The additional actives may include any active ingredients that facilitate oral health, such as actives configured to provide soothing relief, reduce plaque, and/or provide a source of calcium. By way of example, zinc gluconate, zinc chloride, calcium lactate, aloe vera, xylitol, sodium fluoride, potassium nitrate, and/or propanediol (a demulcent) may be an additional active ingredient.

A Composition in accordance with various embodiments of the invention exhibits the surprising and unexpected result of treating various conditions, as set forth herein, without exhibiting metallic or other bad tastes typically associated with other oral rinse compositions or causing tooth discoloration. In addition, the composition allows for a healthy or normal flora to survive in an oral cavity, while treating the conditions noted herein. Furthermore, the compositions are particularly well suited for those suffering from diabetes or prediabetes.

### SPECIFIC EXAMPLES

The following non-limiting examples illustrate exemplary compositions in accordance with various embodiments of the invention. These examples are merely illustrative, and it is not intended that the invention be limited to the examples. Compositions in accordance with the present invention may include the ingredients listed below as well as additional and/or alternative inert materials, preservatives, and other constituents typically found in oral compositions. In the case where exemplary inert materials, preservatives, and/or the like are listed, these ingredients are merely exemplary, and it is understood that other similar ingredients may be substituted for the materials listed in the examples below.

### Example 1

An oral care composition, including the ingredients listed below, is formed by adding water to a mixing tank, and turning on the impeller. The xylitol, aloe vera, zinc chloride, benzoic acid, stevia, disodium phosphate are added to the water and mixed for about thirty minutes to form a first mixture. A portion of the first mixture (e.g., about 10 lbs.) is extracted from the mixing vessel and lactoferrin, lysozyme, papain, and peptizyme are mixed with the extracted formulation until all enzymes are dissolved to form a second mixture. The second mixture is then added back to the first mixture. Concentrated cocamidopropyl betaine is then mixed into the mixture. In a separate vessel, a portion (17%) of the propanediol and the cellulose gum are mixed until all of the gum is dispersed and no lumps are visible. The gum/propanediol mixture is transferred to the mixing tank and mixed into the mixture. The remaining propanediol is then mixed into the mixture. The flavoring is then mixed into the mixture.

The oral composition has a pH between about 4 and about 5.5 and has a specific gravity of about 1.01 to about 1.10. The viscosity of the composition is about 10 to about 60 centipoise.

| | | Exemplary | Exemplary wt |
|---|---|---|---|
| Ingredient | Function | wt % | % range |
| PURIFIED WATER | SOLVENT | 75.487% | 60-90 |
| XYLITOL | SWEETENER | 16.000% | 5-20 |
| ALOE VERA | SOOTHING AGENT | 0.103% | 0.1-2.0 |
| ZINC CHLORIDE | FRESH BREATH | 0.010% | 0.001-0.05 |
| BENZOIC ACID/SODIUM | PRESERVATIVE | 0.010% | 0.005-0.3 |
| BENZOATE | | | |
| STEVIA REBAUDIANA EXTRACT | SWEETENER | 0.030% | 0.01-0.1 |
| DI SODIUM PHOSPHATE | BUFFERING AGENT | 0.010% | 0.005-0.1 |
| LACTOFERRIN | IRON BINDING ENZYME | 0.063% | 0.005-0.3 |
| LYSOZYME | ATTACK CELL WALL OF | 0.063% | 0.005-0.3 |
| | BACTERIA | | |
| PAPAIN | TEETH WHITENING | 0.062% | 0.001-0.1 |
| PEPTIZYME | REDUCES INFLAMMATION | 0.062% | 0.001-0.1 |
| TEGOBETAIN F 50 | HUMECTANT | 0.150% | 0.01-2.0 |
| (Concentrated Cocamidopropyl | | | |
| Betaine) | | | |
| PROPANEDIOL | DEMULCENT | 7.000% | 5-15 |
| CELLULOSE GUM 7H4F | FILM FORMER | 0.800% | 0.05-2.0 |
| CINNAMON FLAVOR CI22743 | FOR FRESHNESS | 0.150% | 0.001-1.0 |
| TOTAL | | 100.000% | |

### Prophetic Example 1

The composition of example 1 is used to treat dry mouth symptoms. The user is instructed to apply about four teaspoons (20 ml) of the composition of example 1 to an oral cavity, swish thoroughly for about 30 seconds to about one minute, and then spit out (discharge) the composition, and to repeat this application 2-3 times per day. The user is further instructed to not immediately rinse the oral cavity after application of the composition or to eat food right after application of the composition. The method of example 2 is thought to reduce dry mouth symptoms.

### Prophetic Example 2

The composition of example 1 is provided to a user with diabetes. The user is instructed to apply about four teaspoons of the composition of example 1 to an oral cavity, swish thoroughly for about 30 seconds to about one minute, then spit out the composition, and to repeat this application 2-3 times per day. The user is further instructed to not immediately rinse the oral cavity or eat food. The user experienced improved oral health, fresher breath, and a reduction in tartar formation.

### Prophetic Example 3

A user applied the composition of example 1 in accordance with the instructions of prophetic examples 2 and 3 and noticed moisturizing and soothing effects of the composition.

### Prophetic Example 4

Another user used the composition of example 1 in accordance with the instructions of prophetic examples 2 and 3. The user experienced improved breath (reduction in bad breath).

### Prophetic Example 5

A user of the composition of example 1 in accordance with the instructions of prophetic examples 2 and 3 noticed an unexpected result that a duration and a severity of his cold was reduced as a result of using the composition of example 1.

### Prophetic Example 6

A user of the composition of example 1 in accordance with the instructions of prophetic examples 2 and 3 noticed an unexpected result that gum disease was reduced.

### Prophetic Example 7

A user of the composition of example 1 in accordance with the instructions of prophetic examples 2 and 3 noticed an unexpected result that periodontal disease was reduced.

### Example 2

A liquid drop formulation is formed using the following ingredients.

| | | Exemplary | Exemplary wt |
|---|---|---|---|
| Ingredient | Function | wt % | % range |
| PURIFIED WATER | SOLVENT | 62.949% | 60-90 |
| XYLITOL | SWEETENER | 20.000% | 5-20 |
| ALOE VERA | SOOTHING AGENT | 0.503% | 0.1-2.0 |
| ZINC CHLORIDE | FRESH BREATH | 0.010% | 0.001-0.05 |
| BENZOIC ACID/SODIUM | PRESERVATIVE | 0.010% | 0.005-0.3 |
| BENZOATE | | | |
| STEVIA REBAUDIANA EXTRACT | SWEETENER | 0.030% | 0.01-0.1 |
| DI SODIUM PHOSPHATE | BUFFERING AGENT | 0.010% | 0.005-0.1 |
| LACTOFERRIN | IRON BINDING ENZYME | 0.063% | 0.005-0.3 |
| LYSOZYME | ATTACK CELL WALL OF | 0.063% | 0.005-0.3 |
| | BACTERIA | | |
| PAPAIN | TEETH WHITENING | 0.062% | 0.001-0.1 |
| PEPTIZYME | REDUCES INFLAMMATION | 0.062% | 0.001-0.1 |
| TEGOBETAIN F 50 | HUMECTANT | 0.150% | 0.01-2.0 |
| (Concentrated Cocamidopropyl | | | |
| Betaine) | | | |
| PROPANEDIOL | DEMULCENT | 14.000% | 5-15 |
| CELLULOSE GUM 7H4F | FILM FORMER | 2.00% | 0.05-2.0 |
| CINNAMON FLAVOR CI22743 | FOR FRESHNESS | 0.150% | 0.001-1.0 |
| TOTAL | | 100.000% | |

### Example 3

A spray formulation is formed using the following ingredients.

| | | Exemplary | Exemplary wt |
|---|---|---|---|
| Ingredient | Function | wt % | % range |
| PURIFIED WATER | SOLVENT | 77.931% | 50-90 |
| XYLITOL | SWEETENER | 12.5% | 5-20 |
| ALOE VERA | SOOTHING AGENT | 0.05% | 0.01-2.0 |
| ZINC CHLORIDE | FRESH BREATH | 0.010% | 0.001-0.05 |
| BENZOIC ACID/SODIUM | PRESERVATIVE | 0.010% | 0.005-0.3 |
| BENZOATE | | | |
| STEVIA REBAUDIANA EXTRACT | SWEETENER | 0.030% | 0.01-0.1 |
| DI SODIUM PHOSPHATE | BUFFERING AGENT | 0.010% | 0.005-0.1 |
| LACTOFERRIN | IRON BINDING ENZYME | 0.063% | 0.005-0.3 |
| LYSOZYME | ATTACK CELL WALL OF | 0.063% | 0.005-0.3 |
| | BACTERIA | | |
| PAPAIN | TEETH WHITENING | 0.062% | 0.001-0.1 |
| PEPTIZYME | REDUCES INFLAMMATION | 0.062% | 0.001-0.1 |
| TEGOBETAIN F 50 | HUMECTANT | 0.001% | 0.0-0.1 |
| (Concentrated Cocamidopropyl | | | |
| Betaine) | | | |
| PROPANEDIOL | DEMULCENT | 8.000% | 5-15 |
| CELLULOSE GUM 7H4F | FILM FORMER | 0.500% | 0.01-1.0 |
| CINNAMON FLAVOR CI22743 | FOR FRESHNESS | 0.150% | 0.001-1.0 |
| TOTAL | | 100.000% | |

### Example 4

A foam formulation is formed using the following ingredients.

| | | Exemplary | Exemplary wt |
|---|---|---|---|
| Ingredient | Function | wt % | % range |
| PURIFIED WATER | SOLVENT | 72.437% | 50-90 |
| XYLITOL | SWEETENER | 16.000% | 5-20 |
| ALOE VERA FROM ALOE CORP | SOOTHING AGENT | 0.103% | 0.01-2.0 |
| ZINC CHLORIDE | FRESH BREATH | 0.010% | 0.001-0.05 |
| BENZOIC ACID/SODIUM | PRESERVATIVE | 0.010% | 0.005-0.3 |
| BENZOATE | | | |
| STEVIA REBAUDIANA EXTRACT | SWEETENER | 0.030% | 0.01-0.1 |
| DI SODIUM PHOSPHATE | BUFFERING AGENT | 0.010% | 0.005-0.1 |
| LACTOFERRIN | IRON BINDING ENZYME | 0.063% | 0.005-0.3 |
| LYSOZYME | ATTACK CELL WALL OF | 0.063% | 0.005-0.3 |
| | BACTERIA | | |
| PAPAIN | TEETH WHITENING | 0.062% | 0.001-0.1 |
| PEPTIZYME | REDUCES INFLAMMATION | 0.062% | 0.001-0.1 |
| TEGOBETAIN F 50 | HUMECTANT | 0.50% | 0.0-2.0 |
| (Concentrated Cocamidopropyl | | | |
| Betaine) | | | |
| PROPANEDIOL | DEMULCENT | 7.000% | 5-15 |
| CELLULOSE GUM 7H4F | FILM FORMER | 0.500% | 0.05-2.0 |
| CINNAMON FLAVOR CI22743 | FOR FRESHNESS | 0.150% | 0.001-1.0 |
| POLOXAMAR407 | SURFACTANT | 3.0% | 0.05-3.0 |
| TOTAL | | 100.000% | |

### Prophetic Example 8

A user of the composition of example 2 in accordance with the instructions of prophetic examples 2 and 3 noticed unexpected results that oral health was improved, bad breath was reduced, dry mouth symptoms were relieved, and plaque, tartar, and gum disease were reduced.

### Prophetic Example 9

A user of the composition of example 10 in accordance with the instructions of prophetic examples 2 and 3 noticed unexpected results that oral health was improved, bad breath was reduced, dry mouth symptoms were relieved, and plaque, tartar, and gum disease were reduced.

### Prophetic Example 10

A user of the composition of example 11 in accordance with the instructions of prophetic examples 2 and 3 noticed unexpected results that oral health was improved, bad breath was reduced, dry mouth symptoms were relieved, and plaque, tartar, and gum disease were reduced.

### Example 5

An oral care composition, including the ingredients listed below, is formed by adding the water to a mixing tank, and turning on the impeller. The sodium benzoate, xylitol, aloe vera, zinc chloride, benzoic acid, stevia, and disodium phosphate are added to the water and mixed for about thirty minutes to form a first mixture. A portion of the first mixture (e.g., about 10 lbs.) is extracted from the mixing vessel and the enzymes are mixed (e.g., thirty minutes or more) with the extracted formulation until all enzymes are dissolved to form a second mixture. The second mixture is then added back to the first mixture. Concentrated cocamidopropyl betaine is then mixed into the mixture. In a separate vessel, a portion (8%) of the propanediol and the cellulose gum are mixed until all of the gum is dispersed and no lumps are visible. The gum/propanediol mixture is transferred to the mixing tank and mixed into the mixture. The remaining propanediol is then mixed into the mixture. The flavoring is then mixed into the mixture.

The oral composition has a pH between about 4.5 and about 6.5 and has a specific gravity of about 1.01 to about 1.10.

| | | Exemplary | Exemplary wt |
|---|---|---|---|
| Ingredient | Function | wt % | % range |
| PURIFIED WATER | SOLVENT | 67.239% | 60-80 |
| SODIUM BENZOATE | PRESERVATIVE | 0.300% | 0.1-1 |
| XYLITOL | SWEETENER/ANTICARIES | 16.000% | 5-20 |
| ALOE VERA | SOOTHING AGENT | 0.051% | 0.01-0.1 |
| ZINC CHLORIDE | ACTIVE | 0.010% | 0.001-0.1 |
| BENZOIC ACID | PRESERVATIVE | 0.010% | 0-0.1 |
| STEVIA | SWEETENER | 0.030% | 0-0.1 |
| DI SODIUM PHOSPHATE | BUFFERING AGENT | 0.010% | 0-0.1 |
| ENZYMES | ACTIVES | 0.250% | 0.1-0.5 |
| TEGOBETAIN F 50 | HUMECTANT | 0.150% | 0-0.5 |
| (Concentrated Cocamidopropyl | | | |
| Betaine) | | | |
| PROPANEDIOL | DEMULCENT | 15.000% | 5-20 |
| CELLULOSE GUM 7H4F | THICKENER | 0.800% | 0-5 |
| CINNAMON FLAVOR CI22743 | FLAVORANT | 0.150% | 0-0.5 |
| TOTAL | | 100.000% | |

### Example 6

An oral care composition, including the ingredients listed below, is formed by adding 12% of the water to a mixing tank, and turning on the impeller. The xylitol, aloe vera, zinc gluconate, benzoic acid, phytic acid, and disodium phosphate are added to the water and mixed for about thirty minutes to form a first mixture. A portion of the first mixture (e.g., about 10 lbs.) is extracted from the mixing vessel and the enzymes are mixed (e.g., thirty minutes or more) with the extracted formulation until all enzymes are dissolved to form a second mixture. The second mixture is then added back to the first mixture. In a separate vessel, a portion (56%) of the propanediol, the xanthan gum, and the cellulose gum are mixed until all of the gum is dispersed and no lumps are visible. The gum/propanediol mixture is transferred to the mixing tank and mixed into the mixture. The remaining propanediol is then mixed into the mixture. The flavoring is then mixed into the mixture.

The oral composition has a pH between about 3 and about 4 and has a specific gravity of about 1.01 to about 1.10.

| | | Exemplary | Exemplary wt |
|---|---|---|---|
| Ingredient | Function | wt % | % range |
| PURIFIED WATER | SOLVENT | 78.064% | 70-80 |
| XYLITOL | SWEETENER | 12.500% | 7-20 |
| PROPANEDIOL | DEMULCENT | 8.860% | 5-10 |
| ALOE VERA | SOOTHING AGENT | 0.050% | 0-0.1 |
| CELLULOSE GUM | THICKENER | 0.100% | 0-0.5 |
| ZINC GLUCONATE | ACTIVE | 0.020% | 0.01-0.5 |
| BENZOIC ACID | PRESERVATIVE | 0.050% | 0-0.1 |
| DISODIUM PHOSPHATE | BUFFERING AGENT | 0.010% | 0-0.1 |
| PHYTIC ACID | CHELATING AGENT | 0.001% | 0-0.1 |
| XANTHAM GUM | THICKENER | 0.050% | 0-0.1 |
| CINNAMON FLAVOR CI 22743 | FLAVORANT | 0.045% | 0-0.1 |
| ENZYMES | ACTIVES | 0.250% | 0.1-0.5 |
| TOTAL | | 100.000% | |

Although exemplary embodiments of the present invention are set forth herein, it should be appreciated that the invention is not so limited. Various modifications, variations, and enhancements in the composition and method set forth herein may be made without departing from the spirit and scope of the present invention. Furthermore, the compositions set forth above may comprise, consist of, or consist essentially of the ingredients noted herein.

What is claimed is:

1. An oral care composition comprising:
a carrier comprising a solvent;
a plurality of enzymes selected from the group consisting of lysozyme, peptizyme, and papain; and
a metal ion management formulation comprising one or more compounds selected from the group consisting of sodium EDTA, phytic acid, and lactoferrin.

2. The oral composition of claim 1, further comprising zinc chloride.

3. The oral composition of claim 1, further comprising a demulcent.

4. The oral composition of claim 1, wherein the solvent comprises purified water, a thickener, and a sweetener.

5. The oral composition of claim 1, wherein the carrier comprises a thickener selected from the group consisting of propanediol, carboxymethyl cellulose, cellulose gum, polyvinyl pyrolidone, hydroxylethyl cellulose, xanthum gum, and combinations thereof.

6. The oral composition of claim 1, wherein the carrier comprises a moisturizing agent.

7. A method of using the oral composition of claim 1, the method comprising the steps of:
applying the composition of claim 1 to an oral cavity; and
discharging the composition.

8. The method of claim 7, wherein the composition is used to freshen breath.

9. The method of claim 7, wherein the composition is used to provide soothing relief.

10. The method of claim 7, wherein the composition is used to provide reduce plaque formation.

11. The method of claim 7, wherein the composition is used to provide reduce tartar formation.

12. The method of claim 7, wherein the composition is used to treat gum disease.

13. The method of claim 7, wherein the composition is used to treat periodontal disease.

14. The method of claim 7, wherein the composition is administered to a person suffering from diabetes or prediabetes.

15. An oral composition consisting essentially of:
a carrier;
a soothing agent;
a demulcent;
zinc chloride;
a plurality of enzymes selected from the group consisting of lysozyme, peptizyme, and papain; and
a metal ion management formulation.

16. An oral composition comprising:
a carrier;
a soothing agent;
a demulcent;
zinc chloride;
a plurality of enzymes, wherein the plurality of enzymes do not break down carbohydrates into sugars; and
a metal ion management formulation.

17. An oral composition comprising:
a carrier;
a soothing agent;
a demulcent;
an anti-tartar agent;
a plurality of enzymes, wherein the plurality of enzymes are not contraindicated for use by diabetics or prediabetics; and
a metal ion management formulation.
